(19) United States
(12) Patent Application Publication
(10) Pub. No.: US 2019/0000739 A1
(43) Pub. Date: Jan. 3, 2019

(54) BLOCK POLYMER BEARING ALKOXYSILANE GROUPS AND COSMETIC USE THEREOF

(71) Applicant: L'OREAL, Paris (FR)

(72) Inventor: Bertrand LION, Aulnay-sous-bois (FR)

(21) Appl. No.: 16/063,557

(22) PCT Filed: Dec. 16, 2016
(86) PCT No.: PCT/EP2016/081343

§ 371 Date: Jun. 18, 2018

(30) Foreign Application Priority Data
Dec. 22, 2015 (FR) 1563106

(51) Int. Cl. A61K 8/58; A61K 8/81; A61Q 1/10; A61Q 5/00

(52) U.S. Cl. CPC A61K 8/585; A61K 8/8164; A61K 2800/95; A61Q 5/00; A61Q 1/10

(57) ABSTRACT

The invention relates to a cosmetic process for caring for or making up keratin materials, comprising: either the topical application to the keratin materials of an anhydrous mixture of a cosmetic composition comprising a maleic anhydride block polymer and of an amino alkoxysilane compound; or the sequential application to the keratin materials of an anhydrous cosmetic composition comprising a maleic anhydride block polymer and of an anhydrous cosmetic composition containing an amino alkoxysilane, said block polymer comprising: a first block with a glass transition temperature (Tg) of greater than or equal to 40° C., obtained from a monomer CH₂═C(R₁)—COOR₂ in which R₁═H or methyl, R₂═C₄ to C₁₂ cycloalkyl group; and a second block with a glass transition temperature (Tg) of less than or equal to 20° C. derived from a maleic anhydride monomer and from a monomer CH₂═C(R₁)—COOR₃ in which R₁═H or methyl, R₃=linear or branched C₁ to C₆ unsubstituted alkyl group, with the exception of a tert-butyl group, or a methoxyethyl group. The invention also relates to the polymer bearing an alkoxysilane group obtained by reacting said block polymer with said amino alkoxysilane. The process makes it possible to obtain a non-tacky and transfer-resistant film-forming deposit that has good persistence and that is resistant to water, to oil and to sebum.

The present invention relates to a block polymer bearing alkoxysilane groups, to a composition comprising such a polymer and to the use of this polymer in the cosmetic field.

Cosmetic products often require the use of a film-forming polymer to obtain a deposit of the product on keratin materials that has good cosmetic properties. In particular, it is necessary for the film-forming deposit to have good persistence, in particular for the deposit not to transfer during contact with the fingers, clothing, a glass or a cup, and also good persistence on contact with water, especially rain or during showering or alternatively perspiration. Skin sebum may also damage the film-forming deposit.

It is known to those skilled in the art to use polymers in order to obtain these properties of good persistence throughout the day. These polymers are of very different chemical natures and are generally conveyed either in a fatty phase or in an aqueous phase. Examples that may be mentioned include silicone resins, polyacrylates and latices.

Although these polymers do indeed afford persistence properties, in particular transfer resistance, they may have a certain level of discomfort: for example, after applying the product, they may have a tacky aspect.

There is thus still a need for polymers that can afford good persistence properties while at the same time maintaining a certain level of comfort during use.

The inventors have discovered that a particular maleic anhydride block polymer combined with a particular aminosilane compound makes it possible to obtain a deposit on keratin materials that has good film-forming properties.

The film-forming deposit obtained has good water resistance and also good resistance to oil (especially to olive oil) and to sebum.

This particular block polymer is readily conveyable in a hydrocarbon-based oil such as isododecane.

Furthermore, the film-forming deposit has good tack-resistance and transfer-resistance properties, especially when the film is touched with the fingers: the deposit obtained thus has good persistence properties.

Furthermore, when the maleic anhydride block polymer is formulated with a non-volatile oil (often used in makeup products), for instance isohexadecane, the process according to the invention makes it possible to obtain a film-forming deposit which has good persistence, transfer-resistance, tack-resistance, water-resistance, oil-resistance and sebum-resistance properties.

This maleic anhydride block polymer combined with said aminosilane forms a film-forming deposit that is suitable for making up the skin or the lips or the eyelashes, such as foundations, lipsticks or mascaras, or for fixing the hair.

More precisely, a subject of the present invention is a process, especially a cosmetic process, for treating and in particular for caring for or making up keratin materials, comprising:

either the topical application to the keratin materials of an anhydrous (extemporaneous) mixture of a cosmetic composition comprising a maleic anhydride block polymer and of an amino alkoxysilane compound (I) or of a cosmetic composition containing same;

or the sequential application to the keratin materials of an anhydrous cosmetic composition comprising a maleic anhydride block polymer and of an amino alkoxysilane compound (I) or of an anhydrous cosmetic composition containing same,

said block polymer comprising:

at least one first block with a glass transition temperature (Tg) of greater than or equal to 40° C. and obtained from at least one (meth)acrylate monomer of formula CH₂═C(R₁)—COOR₂ in which R₁ represents H or a methyl radical, R₂ represents a C₄ to C₁₂ cycloalkyl group; and

at least one second block with a glass transition temperature (Tg) of less than or equal to 20° C. and obtained from at least one maleic anhydride monomer and from at least one (meth)acrylate monomer of formula CH₂═C(R₁)—COOR₃ in which R₁ represents H or a methyl radical, R₃ representing a linear or branched C₁ to C₆ unsubstituted alkyl group, with the exception of a tert-butyl group, or a methoxyethyl group;

said amino alkoxysilane having the formula (I):

R—NH—R₁Si(OR₂)_{z}(R₃)ₓ (I)

in which:

R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;

R═H or a C₁-C₄ alkyl group, preferably H;

R₂ and R₃, which may be identical or different, represent a linear or branched alkyl group comprising from 1 to 6 carbon atoms,

z denotes an integer ranging from 1 to 3, and

x denotes an integer ranging from 0 to 2,

with z+x=3.

The process according to the invention is suitable for caring for or making up keratin materials, such as the skin, the lips, the eyelashes or the nails.

According to one embodiment of the process according to the invention, an extemporaneous mixture of an anhydrous composition comprising a maleic anhydride block polymer and of an amino alkoxysilane (I), or an anhydrous composition containing same and comprising a physiologically acceptable medium, as defined below, is applied topically to keratin materials.

According to another embodiment of the process according to the invention, an anhydrous composition comprising a maleic anhydride block polymer as described previously and an amino alkoxysilane (I), or an anhydrous composition containing same and comprising a physiologically acceptable medium, as defined previously, are applied sequentially to keratin materials.

A subject of the invention is also a composition, especially a cosmetic composition, obtained by mixing an anhydrous composition comprising said maleic anhydride block polymer and an amino alkoxysilane (I) or an anhydrous composition containing same and comprising a physiologically acceptable medium, as defined below.

A subject of the invention is also a kit comprising a first anhydrous composition comprising said maleic anhydride block polymer as described previously and a second anhydrous composition comprising an amino alkoxysilane (I) as described previously and comprising a physiologically acceptable medium, the first and second compositions each being packaged in a separate packaging assembly.

The composition packaging assembly is, in a known manner, any packaging that is suitable for storing cosmetic compositions (especially a bottle, tube, spray bottle or aerosol bottle).

Such a kit allows the skin treatment process according to the invention to be performed.

The block polymer used according to the invention comprises:

at least one first block with a glass transition temperature (Tg) of greater than or equal to 40° C. and obtained from at least one (meth)acrylate monomer of formula CH₂═C(R₁)—COOR₂ in which R₁ represents H or a methyl radical, R₂ represents a C₄ to C₁₂ cycloalkyl group; and

at least one second block with a glass transition temperature (Tg) of less than or equal to 20° C. and obtained from at least one maleic anhydride monomer and from at least one (meth)acrylate monomer of formula CH₂═C(R₁)—COOR₃ in which R₁ represents H or a methyl radical, R₃ representing a linear or branched C₁ to C₆ unsubstituted alkyl group, with the exception of a tert-butyl group, or a methoxyethyl group.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

1/Tg=Σ(ωᵢ/Tgᵢ),

i

ωᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The difference between the glass transition temperatures of the first and second blocks is generally greater than 20° C., preferably greater than 40° C. and better still greater than 60° C.

In the present invention, the expression:

“between . . . and . . . ” means a range of values in which the limits mentioned are excluded, and

“from . . . to . . . ” and “ranging from . . . to . . . ” mean a range of values in which the limits are included.

The block polymer used according to the invention has a first block with a glass transition temperature (Tg) of greater than or equal to 40° C., for example a Tg ranging from 40 to 150° C., and obtained from at least one (meth)acrylate monomer of formula CH₂═C(R₁)—COOR₂ in which R₁ represents H or a methyl radical, R₂ represents a C₄ to C₁₂ cycloalkyl group; and preferably an isobornyl group.

Preferably, said first block has a Tg of greater than or equal to 60° C., ranging, for example, from 60° C. to 140° C., especially ranging from 80° C. to 120° C., preferentially ranging from 95 to 110° C.

The monomers present in the first block of the polymer and the proportions thereof are preferably chosen such that the glass transition temperature of the first block is greater than or equal to 40° C., and especially in accordance with that described previously.

According to a preferred embodiment, the first block of the polymer is obtained from at least one acrylate monomer of formula CH₂═CH—COOR₂ in which R₂ represents a C₄ to C₁₂ cycloalkyl group, and from at least one methacrylate monomer of formula CH₂═C(CH₃)—COOR′₂ in which R′₂ represents a C₄ to C₁₂ cycloalkyl group.

The first block of the polymer may be obtained exclusively with said acrylate monomer and said methacrylate monomer.

The acrylate monomer and the methacrylate monomer used are preferably present in acrylate/methacrylate mass proportions of between 30/70 and 70/30, preferably between 40/60 and 60/40 and in particular between 45/55 and 55/45.

The proportion of the first block in the block polymer advantageously ranges from 60% to 80% and better still from 65% to 75% by weight of the polymer.

According to a preferred embodiment, the first block of the polymer is obtained by polymerization of isobornyl methacrylate and isobornyl acrylate.

The first block of the polymer may also comprise an additional monomer chosen from linear or branched C₈-C₂₂ alkyl (meth)acrylates (i.e. comprising a C₈-C₂₂ alkyl group), for instance 2-ethylhexyl acrylate, 2-ethylhexyl methacrylate, lauryl acrylate, lauryl methacrylate, behenyl acrylate, behenyl methacrylate, stearyl acrylate and stearyl methacrylate.

Said additional monomer may be present in a content ranging from 0.1% to 15% by weight and preferably ranging from 0.1% to 5% by weight, relative to the total weight of the monomers of the first block of said block polymer.

According to one embodiment, the first block of said block polymer does not contain any additional monomer.

The block polymer used according to the invention has a second block with a glass transition temperature (Tg) of less than or equal to 20° C., for example a Tg ranging from −100 to 20° C., and is obtained from at least one maleic anhydride monomer and from at least one (meth)acrylate monomer of formula CH₂═C(R₁)—COOR₃ in which R₁ represents H or a methyl radical, R₃ representing a linear or branched C₁ to C₆ unsubstituted alkyl group, with the exception of a tert-butyl group, or a methoxyethyl group.

Preferably, said second block has a Tg of less than or equal to 10° C., especially ranging from −80° C. to 10° C. and better still less than or equal to 0° C., for example ranging from −100° C. to 0° C., especially ranging from −30° C. to 0° C.

The monomers present in the second block of the polymer and the proportions thereof are preferably chosen such that the glass transition temperature of the second block is less than or equal to 20° C., and especially in accordance with that described previously.

The preferred monomers with a Tg of less than or equal to 20° C. are isobutyl acrylate, ethyl acrylate, n-butyl acrylate and methoxyethyl acrylate, or mixtures thereof in all proportions, and preferably isobutyl acrylate.

The second block of the polymer may be obtained exclusively with maleic anhydride and said (meth)acrylate monomer.

In the second block, the maleic anhydride and the (meth)acrylate monomer are preferably used in (meth)acrylate/maleic anhydride mass proportions ranging from 1 to 10, preferentially ranging from 2 to 9, especially ranging from 3 to 8 or alternatively ranging from 4 to 7.

The proportion of the second block in the block polymer advantageously ranges from 20% to 40% and better still from 25% to 35% by weight of the polymer.

According to a preferred embodiment, the second block of the polymer is obtained by polymerization of maleic anhydride and isobutyl acrylate.

The second block of the polymer may also comprise an additional silicone monomer of formula (II) (referred to hereinbelow as a silicone monomer) below:

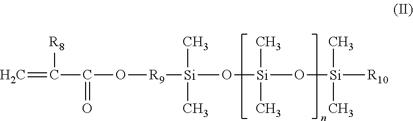

in which:
R₈ denotes a hydrogen atom or a methyl group; preferably methyl;
R₉ denotes a linear or branched, preferably linear, divalent hydrocarbon-based group containing from 1 to 10 carbon atoms, preferably containing from 2 to 4 carbon atoms, and optionally containing one or two —O— ether bonds; preferably an ethylene, propylene or butylene group;
R₁₀ denotes a linear or branched alkyl group containing from 1 to 10 carbon atoms, especially from 2 to 8 carbon atoms; preferably methyl, ethyl, propyl, butyl or pentyl;
n denotes an integer ranging from 1 to 300, preferably ranging from 3 to 200 and preferentially ranging from 5 to 100.

Monomer (II) is a polydimethylsiloxane bearing a mono(meth)acryloyloxy end group.

Use may be made in particular of monomethacryloyloxypropyl polydimethylsiloxanes such as those sold under the names MCR-M07, MCR-M17, MCR-M11 and MCR-M22 by Gelest Inc. or the silicone macromonomers sold under the names X-22-2475, X-22-2426 and X-22-174DX by Shin-Etsu.

Monomer (II) may be present in the second block of the block polymer in a content ranging from 0.1% to 15% by weight, relative to the total weight of the monomers of the second block of said block polymer, and preferably ranging from 0.1% to 5%.

According to one embodiment, the second block of said block polymer does not contain any additional monomer.

Preferably, the polymer used according to the invention comprises at least, or even consists of, isobornyl acrylate and isobornyl methacrylate monomers in the first block and maleic anhydride and isobutyl acrylate monomers in the second block.

Preferably, the polymer comprises at least, or even consists of, isobornyl acrylate and isobornyl methacrylate monomers in a mass proportion ranging from 30/70 to 70/30 in the first block and isobutyl acrylate and maleic anhydride monomers in the second block.

Preferably, the polymer comprises at least, or even consists of, isobornyl acrylate and isobornyl methacrylate monomers in a mass proportion ranging from 40/60 to 60/40 in the first block and isobutyl acrylate and maleic anhydride monomers in the second block.

Preferably, the polymer comprises at least, or even consists of, isobornyl acrylate and isobornyl methacrylate monomers in a mass proportion ranging from 45/55 to 55/45 in the first block and isobutyl acrylate and maleic anhydride monomers in the second block.

Preferably, the polymer comprises at least, or even consists of, isobornyl acrylate and isobornyl methacrylate monomers in a mass proportion ranging from 30/70 to 70/30 in the first block and isobutyl acrylate and maleic anhydride monomers in the second block, the first block representing between 65% and 75% by weight of the polymer, and especially 70% by weight.

Preferably, the polymer comprises at least, or even consists of, isobornyl acrylate and isobornyl methacrylate monomers in a mass proportion ranging from 40/60 to 60/40 in the first block and isobutyl acrylate and maleic anhydride monomers in the second block, the first block representing between 65% and 75% by weight of the polymer, and especially 70% by weight.

Preferably, the polymer comprises at least, or even consists of, isobornyl acrylate and isobornyl methacrylate monomers in a mass proportion ranging from 45/55 to 55/45 in the first block and isobutyl acrylate and maleic anhydride monomers in the second block, the first block representing between 65% and 75% by weight of the polymer, and especially 70% by weight.

Preferably, the polymer comprises at least, or even consists of, isobornyl acrylate and isobornyl methacrylate monomers in a mass proportion ranging from 30/70 to 70/30 in the first block and isobutyl acrylate and maleic anhydride monomers in the second block, the first block representing between 65% and 75% by weight of the polymer, and especially 70% by weight, and the maleic anhydride representing from 3% to 7% by weight of the polymer.

Preferably, the polymer comprises at least, or even consists of, isobornyl acrylate and isobornyl methacrylate monomers in a mass proportion ranging from 40/60 to 60/40 in the first block and isobutyl acrylate and maleic anhydride monomers in the second block, the first block representing between 65% and 75% by weight of the polymer, and especially 70% by weight, and the maleic anhydride representing from 3% to 7% by weight of the polymer.

Preferably, the polymer comprises at least, or even consists of, isobornyl acrylate and isobornyl methacrylate monomers in a mass proportion ranging from 45/55 to 55/45 in the first block and isobutyl acrylate and maleic anhydride monomers in the second block, the first block representing between 65% and 75% by weight of the polymer, and especially 70% by weight, and the maleic anhydride representing from 3% to 7% by weight of the polymer.

Said first and second blocks of the polymer may be advantageously linked together via an intermediate block comprising at least one constituent monomer of the first block and at least one constituent monomer of the second block.

The intermediate segment is a block comprising at least one constituent monomer of the first block and at least one constituent monomer of the second block of the polymer, which enables these blocks to be “compatibilized”.

Advantageously, the intermediate segment comprising at least one constituent monomer of the first block and at least one constituent monomer of the second block of the polymer is a statistical polymer.

Preferably, the intermediate segment is derived essentially from constituent monomers of the first block and of the second block.

The term “essentially” means at least 85%, preferably at least 90%, better still 95% and even better still 100%.

Advantageously, the intermediate block has a glass transition temperature Tg that is between the glass transition temperatures of the first and second blocks.

The block polymer used according to the invention is advantageously a film-forming polymer. The term “film-forming polymer” means a polymer that is capable of forming, by itself or in the presence of a film-forming auxiliary agent, a continuous film that adheres to a support, especially to keratin materials.

Advantageously, the block polymer has a polydispersity index of greater than 2.

The polydispersity index I of the polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector).

The weight-average mass (Mw) of the block polymer is preferably less than or equal to 300 000; it ranges, for example, from 35 000 to 200 000 and better still from 45 000 to 150 000 g/mol.

The number-average mass (Mn) of the block polymer is preferably less than or equal to 70 000; it ranges, for example, from 10 000 to 60 000 and better still from 12 000 to 50 000 g/mol.

Preferably, the polydispersity index of the block polymer is greater than 2, for example ranging from 3 to 11, preferably greater than or equal to 4, for example ranging from 4 to 10.

A subject of the invention is also a process for preparing a block polymer, which consists in mixing, in the same reactor, a polymerization solvent, an initiator, a maleic anhydride monomer, at least one (meth)acrylate monomer of formula CH₂═C(R₁)—COOR₃ in which R₁ represents H or a methyl radical, R₃ represents a linear or branched C₁ to C₆ unsubstituted alkyl group, with the exception of a tert-butyl group, or a methoxyethyl group, at least one (meth)acrylate monomer of formula CH₂═C(R₁)—COOR₂ in which R₁ represents H or a methyl radical, R₂ represents a C₄ to C₁₂ cycloalkyl group, according to the following sequence of steps:
some of the polymerization solvent and some of the initiator are poured into the reactor, and the mixture is heated to a reaction temperature of between 60° C. and 120° C.,
said at least one (meth)acrylate monomer of formula CH₂═C(R₁)—COOR₂ is then poured in, as a first addition, and the mixture is left to react for a time T corresponding to a maximum degree of conversion of said monomers of 90%,
further polymerization initiator, the maleic anhydride monomer and said (meth)acrylate of formula CH₂═C(R₁)—COOR₃ are then poured into the reactor, as a second addition, and the mixture is left to react for a time T′ after which the degree of conversion of said monomers reaches a plateau,
the reaction mixture is cooled to room temperature.

The term “polymerization solvent” means a solvent or a mixture of solvents. The polymerization solvent may be chosen especially from ethyl acetate, butyl acetate, C₈-C₁₆ branched alkanes such as C₈-C₁₆ isoalkanes, for instance isododecane, isodecane or isohexadecane, and mixtures thereof. Preferably, the polymerization solvent is isododecane.

According to another embodiment, a subject of the invention is a process for preparing a polymer, which consists in mixing, in the same reactor, a polymerization solvent, an initiator, a maleic anhydride monomer, at least one (meth)acrylate monomer of formula CH₂═C(R₁)—COOR₃ in which R₁ represents H or a methyl radical, R₃ represents a linear or branched C₁ to C₆ unsubstituted alkyl group, with the exception of a tert-butyl group, or a methoxyethyl group, at least one (meth)acrylate monomer of formula CH₂═C(R₁)—COOR₂ in which R₁ represents H or a methyl radical, R₂ represents a C₄ to C₁₂ cycloalkyl group, according to the following sequence of steps:
some of the polymerization solvent and some of the initiator are poured into the reactor, and the mixture is heated to a reaction temperature of between 60° C. and 120° C.,
the maleic anhydride monomer and said (meth)acrylate of formula CH₂═C(R₁)—COOR₃ are then poured in, as a first addition, and the mixture is left to react for a time T corresponding to a maximum degree of conversion of said monomers of 90%,
further polymerization initiator and said at least one (meth)acrylate of formula CH₂═C(R₁)—COOR₂ are then poured into the reactor, as a second addition, and the mixture is left to react for a time T′ after which the degree of conversion of said monomers reaches a plateau,
the reaction mixture is cooled to room temperature.

The polymerization temperature is preferably between 85 and 95° C., especially about 90° C.

The reaction time after the second addition is preferably between 3 and 6 hours.

The monomers used in the context of this process, and the proportions thereof, may be those described previously.

The polymerization is especially performed in the presence of a radical initiator especially of peroxide type (for example tert-butyl peroxy-2-ethylhexanoate: Trigonox 21S; 2,5-dimethyl-2,5-bis(2-ethylhexanoylperoxy)hexane:Trigonox 141; tert-butyl peroxypivalate: Trigonox 25C75 from AkzoNobel) or of azo type, for example (AlBN: azobisisobutyronitrile; V50: 2,2′-azobis(2-amidinopropane) dihydrochloride).

A subject of the invention is also, as novel polymer, the block polymer described previously.

The polymer used according to the invention may be used in an anhydrous composition comprising a physiologically acceptable medium, in particular in a cosmetic composition.

The term “physiologically acceptable medium” means a medium that is compatible with human keratin materials and in particular with the skin.

The term “cosmetic composition” is understood to mean a composition that is compatible with keratin materials, which has a pleasant colour, odour and feel and which does not cause unacceptable discomfort (stinging, tautness or redness) liable to discourage the consumer from using it.

The maleic anhydride block polymer as defined previously may be present in the composition used according to the invention in a content ranging from 0.1% to 40% by weight, relative to the total weight of the composition derived from the extemporaneous mixture, preferably from 0.5% to 35% by weight, preferentially ranging from 1% to 30% by weight and more preferentially ranging from 10% to 30% by weight. This is the composition that is applied to the keratin materials.

For the amino alkoxysilane of formula (I) used in the process according to the invention: preferably, R₂ represents an alkyl group comprising from 1 to 4 carbon atoms.

Preferably, R₂ represents a linear alkyl group comprising from 1 to 4 carbon atoms.

Preferably, R₂ represents an ethyl group.

Preferably, R₃ represents an alkyl group comprising from 1 to 4 carbon atoms.

Preferably, R₃ represents a linear alkyl group comprising from 1 to 4 carbon atoms.

Preferably, R₃ represents a methyl or ethyl group.

Preferably, R═H.

Preferably, R₁ is an acyclic chain.

Preferably, R₁ is a linear or branched, saturated or unsaturated C₁-C₆ hydrocarbon-based chain. Preferentially, R₁ is a linear saturated C₁-C₆ hydrocarbon-based chain. More preferentially, R₁ is a linear saturated C₂-C₄ hydrocarbon-based chain.

Preferably, R₁ is a substituted linear saturated C₁-C₆ hydrocarbon-based chain.

R═H,

R₂ represents an alkyl group comprising from 1 to 4 carbon atoms,

R₃ represents an alkyl group comprising from 1 to 4 carbon atoms.

Preferably, z is equal to 3.

Preferably, the amino alkoxysilane of formula (I) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

Preferably, the amino alkoxysilane (I) is chosen from 3-am inopropyltriethoxysilane (APTES), 3-aminoethyltriethoxysilane (AETES), 3-aminopropylmethyldiethoxysilane and N-(2-aminoethyl)-3-aminopropyltriethoxysilane.

Preferably, the amino alkoxysilane (I) is 3-aminopropyltriethoxysilane (APTES).

Advantageously, the amino alkoxysilane is used in a mole ratio of amino alkoxysilane/maleic anhydride group of the acrylic polymer ranging from 0.01 to 10, preferably ranging from 0.1 to 5, preferentially ranging from 0.1 to 2 and more preferentially ranging from 0.1 to 1.

The amino alkoxysilane (I) used reacts with the maleic anhydride group present in the block polymer to form a unit having the following formula:

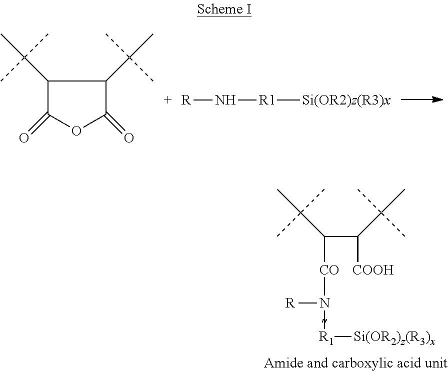

Such a block polymer bearing an amino alkoxysilane group is novel and thus also forms the subject of the present invention. A subject of the invention is also an anhydrous composition comprising such a polymer bearing an amino alkoxysilane group and a physiologically acceptable medium.

The block polymer bearing an amino alkoxysilane group may thus be obtained by reacting the amino alkoxysilane (I) with the maleic anhydride block polymer described previously. Some or all of the anhydride groups react with the NH group of compound (I) and form a unit bearing an amide group and a carboxylic acid group as described in scheme I.

According to one embodiment of the process according to the invention, a mixture, especially an extemporaneous mixture, of the maleic anhydride block polymer and of an amino alkoxysilane (I) is applied to the keratin materials, in particular to the skin. It is also possible to perform sequential application of, on the one hand, the maleic anhydride block polymer and, on the other hand, an amino alkoxysilane (I) as defined previously.

Advantageously, the process according to the invention is performed under ambient conditions, in particular at an ambient temperature that may range from 15° C. to 30° C., preferably ranging from 18° C. to 25° C.

The composition used according to the invention is generally suitable for topical application to keratin materials, in particular to the skin, and thus generally comprises a physiologically acceptable medium, i.e. a medium that is compatible with the skin and/or its integuments. It is preferably a cosmetically acceptable medium, i.e. a medium which has a pleasant colour, odour and feel and which does not cause any unacceptable discomfort (stinging, tautness or redness) liable to discourage the consumer from using this composition.

According to a preferred embodiment of the invention, the composition comprising the maleic anhydride block polymer may contain a hydrocarbon-based oil.

The hydrocarbon-based oil is an oil that is liquid at room temperature (25° C.).

The term “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

The hydrocarbon-based oil may be volatile or non-volatile.

The hydrocarbon-based oil may be chosen from:

hydrocarbon-based oils containing from 8 to 14 carbon atoms, and especially:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade name Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in Examples 1 and 2 of patent application WO 2008/155 059 from the company Cognis, and mixtures thereof,
   short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have chain lengths varying from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stéarineries Dubois or those sold under the names Miglyol 810®, 812® and 818® by the company Dynamit Nobel,
synthetic ethers having from 10 to 40 carbon atoms;
linear or branched hydrocarbons of mineral or synthetic origin, such as petroleum jelly, polydecenes, hydrogenated polyisobutene such as Parleam®, squalane and liquid paraffins, and mixtures thereof,
synthetic esters such as oils of formula R₁COOR₂ in which R₁ represents a linear or branched fatty acid residue containing from 1 to 40 carbon atoms and R₂ represents an, in particular, branched hydrocarbon-based chain containing from 1 to 40 carbon atoms, on condition that R₁+R₂≥10, for instance purcellin oil (cetostearyl octanoate), isopropyl myristate, isopropyl palmitate, C₁₂ to C₁₅ alcohol benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol,

Advantageously, the hydrocarbon-based oil is apolar (thus formed solely from carbon and hydrogen atoms).

The hydrocarbon-based oil is preferably chosen from hydrocarbon-based oils containing from 8 to 14 carbon atoms, in particular the apolar oils described previously.

Preferentially, the hydrocarbon-based oil is isododecane.

The composition comprising the polymer may contain, in addition to the hydrocarbon-based oil, a silicone oil. The term “silicone oil” means an oil comprising at least one silicon atom and especially at least one Si—O group. The silicone oil may be volatile or non-volatile.

The term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and at atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

The term “non-volatile oil” means an oil with a vapour pressure of less than 0.13 Pa.

Volatile silicone oils that may be mentioned include volatile linear or cyclic silicone oils, especially those with a viscosity 8 centistokes (cSt) (8×10⁻⁶ m²/s), and especially having from 2 to 10 silicon atoms and in particular from 2 to 7 silicon atoms, these silicones optionally comprising alkyl or alkoxy groups having from 1 to 10 carbon atoms. As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As non-volatile silicone oils, mention may be made of linear or cyclic non-volatile polydimethylsiloxanes (PDMSs); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

Advantageously, the composition may comprise a hydrocarbon-based oil in a content ranging from 60% to 100% by weight relative to the total weight of the oils present in the composition and from 0 to 40% by weight of silicone oil. According to a preferred embodiment of the invention, the composition contains as oil only a hydrocarbon-based oil.

The composition according to the invention may comprise a cosmetic additive chosen from fragrances, preserving agents, fillers, UV-screening agents, oils, waxes, surfactants, moisturizers, vitamins, ceramides, antioxidants, free-radical scavengers, polymers, thickeners and dyestuffs.

The composition according to the invention may also comprise a dyestuff such as pulverulent dyestuffs, liposoluble dyes or water-soluble dyes. This dyestuff may be present in a content ranging from 0.01% to 30% by weight, relative to the total weight of the composition.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium, zinc or cerium oxide, and also iron or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

Advantageously, the composition according to the invention is a skincare composition.

The composition according to the invention may be a makeup composition such as a foundation, a lipstick or an eye liner.

According to one embodiment, the composition according to the invention is a makeup composition and comprises a volatile oil and a non-volatile oil as described previously. In particular, the makeup composition may comprise a hydrocarbon-based volatile oil and a hydrocarbon-based non-volatile oil.

According to one embodiment, the composition according to the invention is an anhydrous composition. The term “anhydrous composition” means a composition containing less than 2% by weight of water, or even less than 0.5% of water, and especially free of water. Where appropriate, such small amounts of water may especially be introduced by ingredients of the composition that may contain residual amounts thereof.

Needless to say, those skilled in the art will take care to select this or these optional additional compounds, and/or the amount thereof, such that the anti-wrinkle properties of the composition according to the invention are not, or are not substantially, adversely affected by the envisaged addition.

According to a first embodiment of the process according to the invention, a mixture, especially an extemporaneous mixture, of an anhydrous cosmetic composition comprising the maleic anhydride block polymer and of an amino alkoxysilane (I), as previously described, is applied to the keratin materials, especially to the skin. The extemporaneous mixture is advantageously prepared less than 5 minutes before it is applied to the keratin materials, especially to the skin, and preferably less than 3 minutes.

According to a second embodiment of the process according to the invention, an anhydrous composition, especially a cosmetic anhydrous composition, comprising the block polymer is first applied to the keratin materials, especially to the skin, and an amino alkoxysilane (I) or an anhydrous cosmetic composition containing same is then applied. The application of the amine compound may be performed after a time of between 5 minutes and one hour after having applied the acrylic polymer to the keratin materials, especially to the skin.

According to a third embodiment of the process according to the invention, the amino alkoxysilane (I), or a cosmetic anhydrous composition containing same, is first applied to the keratin materials, especially to the skin, and the anhydrous composition, especially cosmetic anhydrous composition, comprising the block polymer is then applied. The application of the block polymer may be performed after a time of between 5 minutes and one hour after having applied the amino alkoxysilane to the keratin materials, especially to the skin.

The application of the cosmetic composition used according to the invention is performed according to the usual techniques, for example by application (in particular of creams, gels, sera or lotions) to the skin intended to be treated, in particular facial and/or neck skin, especially the skin of the area around the eyes. In the context of this process, the composition may, for example, be a care composition.

The invention will now be described with reference to the following examples, which are given as non-limiting illustrations.

### EXAMPLE 1

### Isobornyl methacrylate/isobornyl acrylate (35/35 by weight)-co-isobutyl acrylate/maleic anhydride (25/5 by weight) copolymer

1 litre of isododecane was placed in a jacketed 1-litre reactor equipped with a stirring anchor and the temperature was then increased from 25° C. to 90° C. over 1 hour

105 g of isobornyl methacrylate, 105 g of isobornyl acrylate and 1.8 g of 2,5-bis(2-ethylhexanoylperoxy)-2,5-dimethylhexane (Trigonox® 141 from AkzoNobel) were then added over 1 hour. The reaction mixture was stirred for 1 hour 30 minutes at 90° C.

75 g of isobutyl acrylate, 15 g of maleic anhydride and 1.2 g of 2,5-bis(2-ethylhexanoylperoxy)-2,5-dimethylhexane were then added over 30 minutes.

The reaction mixture was stirred for 3 hours at 90° C. and was then cooled to room temperature (25° C.) and diluted by adding 150 g of isododecane.

A solution containing 50% polymer active material in isododecane was thus obtained.

The polymer obtained has a number-average molecular weight (Mn) of 18 000 and a weight-average molecular weight (Mw) of 290 200; with an Ip=8.9.

### COMPARATIVE EXAMPLES 2 TO 7

### Cosmetic Evaluation of Makeup Compositions with Application in Two Steps

The three base coat makeup compositions (lip gloss; foundation) and a top coat composition containing APTES described below were prepared.

Each base coat composition was applied onto a skin equivalent support made of elastomer by producing a deposit with a wet thickness of 100 μm, which was left to dry at room temperature (25° C.) for 24 hours.

The top coat composition was then applied onto each dry base coat deposit by producing a deposit with a wet thickness of 100 μm, which was left to dry at room temperature (25° C.) for 24 hours.

The state of the film obtained before (outside the invention) and after (invention) applying the top coat composition was then observed.

The resistance of the film obtained was evaluated by separately applying 0.5 ml of water, 0.5 ml of olive oil and 0.5 ml of sebum; after 5 minutes of contact, the surface of the film was rubbed with cotton wool and the state of the film was then observed (degraded or undegraded appearance of the film).

The tackiness of the film and its capacity for transferring or not transferring on touching the film with a finger were also evaluated.

The evaluation was made in the following manner:

+++: very efficient evaluated cosmetic property

++: moderately efficient evaluated cosmetic property

+: sparingly efficient evaluated cosmetic property

0: inefficient evaluated cosmetic property

The following results were obtained:

| | | Example 3 | | Example 5 |
|---|---|---|---|---|
| | Example 2 | (invention) | Example 4 | (invention) |
| Base Coat | | | | |
| Polymer of | 25 g AM | 25 g AM | 20 g AM | 20 g AM |
| Example 1 | | | | |
| Pigmentary paste | 5 g | 5 g | 5 g | 5 g |
| containing 40% by | with DC | with DC | with DC | with DC |
| weight of pigment | Red 7 | Red 7 | Red 7 | Red 7 |
| in isododecane | | | | |
| Disteardimonium | 10 g | 10 g | 10 g | 10g |
| hectorite | | | | |
| (Bentone Gel ISD V | | | | |
| from Elementis) | | | | |
| Isohexadecane | | | 40 g | 40 g |
| Isododecane | qs 100 g | qs 100 g | qs 100 g | qs 100 g |
| Top Coat | | | | |
| APTES | | 5 g | | 5 g |
| Isododecane | | 95 g | | 95 g |
| Evaluation of | | | | |
| the film | | | | |
| Appearance of | Homo- | Homo- | Homo- | Homo- |
| the film | geneous | geneous | geneous | geneous |
| | film | film | film | film |
| Water resistance | ++ | +++ | ++ | +++ |
| Olive oil | + | +++ | + | +++ |
| resistance | | | | |
| Sebum resistance | + | +++ | + | +++ |
| Non-tacky | + | +++ | 0 | +++ |
| Transfer-resistant | + | +++ | 0 | +++ |

| | | Example 7 |
|---|---|---|
| | Example 6 | (invention) |
| Base Coat | | |
| Polymer of | 25 g AM | 25 g AM |
| Example 1 | | |
| Pigmentary paste | 5 g | 5 g |
| containing 40% by | with red | with red |
| weight of pigment | iron oxide | iron oxide |
| in isododecane | | |
| Disteardimonium | 10 g | 10 g |
| hectorite | | |
| (Bentone Gel ISD V | | |
| from Elementis) | | |
| Isododecane | 65 g | 65 g |
| Top Coat | | |
| APTES | | 15 g |
| Isododecane | | 95 g |
| Evaluation of | | |
| the film | | |
| Appearance of | Homo- | Homo- |
| the film | geneous | geneous |
| | film | film |
| Water resistance | ++ | +++ |
| Olive oil | + | +++ |
| resistance | | |
| Sebum resistance | + | +++ |
| Non-tacky | + | +++ |
| Transfer-resistant | + | +++ |

The results obtained show that the deposits resulting from the application of polymer 1, with or without isohexadecane, followed by APTES (Examples 3, 5; 7) form a non-tacky homogeneous film that does not transfer to the finger, and that is resistant to water, to oil and to sebum, whereas the application of polymer 1 alone (Examples 2, 4; 6) forms a deposit that is much more tacky and that transfers onto the finger and has poorer resistance to water, to oil and to sebum.

Thus, the non-tacky aspect and transfer-resistant aspect on contact with the finger, and also the resistance of the film to contact with olive oil and sebum are markedly improved with the application of the top coat composition containing APTES.

The lipstick compositions of Examples 3 and 5 applied to the lips thus make it possible to obtain a non-tacky, transfer-resistant and water-, oil- and sebum-resistant makeup which thus has good persistence.

The compositions of Example 7 applied to the skin thus make it possible to obtain a non-tacky, transfer-resistant and water-, oil- and sebum-resistant makeup which thus has good persistence.

### COMPARATIVE EXAMPLES 8 AND 9

### Cosmetic Evaluation of Makeup Compositions with Application in One Step

The makeup (lip gloss) compositions described below containing the polymer of Example 1 with or without APTES were prepared, and the composition was then applied onto a skin equivalent support made of elastomer by producing a deposit with a wet thickness of 100 μm, which was left to dry at room temperature (25° C.) for 24 hours.

The cosmetic properties of the film obtained were evaluated according to the tests described previously in Examples 2 to 7.

The following results were obtained:

| | | Example 9 |
|---|---|---|
| | Example 8 | (invention) |
| Composition | | |
| Polymer of | 25 g AM | 25 g AM |
| Example 1 | | |
| Pigmentary paste | 5 g | 5 g |
| containing 40% by | with DC | with DC |
| weight of pigment | Red 7 | Red 7 |
| in isododecane | | |
| Disteardimonium | 10 g | 10 g |
| hectorite | | |
| (Bentone Gel ISD V | | |
| from Elementis) | | |
| Isododecane | 65 g | 65 g |
| Evaluation of | | |
| the film | | |
| Appearance of | Homo- | Homo- |
| the film | geneous | geneous |
| | film | film |
| Water resistance | ++ | +++ |
| Olive oil | + | +++ |
| resistance | | |
| Sebum resistance | + | +++ |
| Non-tacky | + | +++ |
| Transfer-resistant | + | +++ |

The results obtained show that the deposit resulting from the application of polymer 1 mixed with APTES (Example 9) forms a non-tacky homogeneous film that does not transfer to the finger, and that is resistant to water, to oil and to sebum, whereas the application of polymer 1 alone (Example 8) forms a deposit that is much more tacky and that transfers onto the finger and has poorer resistance to water, to oil and to sebum.

Thus, the non-tacky and transfer-resistant aspect on contact with the finger, and also the resistance of the film to contact with water, olive oil and sebum, are markedly improved with the application of the composition containing the polymer of Example 1 and APTES.

The lipstick compositions of Example 9 applied to the lips thus make it possible to obtain a non-tacky, transfer-resistant and water-, oil- and sebum-resistant makeup which thus has good persistence.

### COMPARATIVE EXAMPLES 10 TO 12

### Cosmetic Evaluation of a Hair Composition

0.5 g of a base coat composition containing 10% AM of the polymer of Example 1 in isododecane was applied to a 2.7 g lock of washed and dried hair (Lock No. 1). The treated lock was left to dry naturally (25° C.) for 24 hours. 0.5 g of a top coat composition containing 10% AM of APTES in isododecane was then applied to the lock, which was then left to dry naturally for 24 hours (Example 12 according to the invention). The base coat composition alone was applied to another lock of hair (Lock No. 2) (Example 10). The top coat composition alone was applied to another lock of hair (Lock No. 3) (Example 11).

The fixing quality of the lock of hair was evaluated by observing the more or less rigid appearance of the lock: the lock is taken by one of its ends with the fingers and turned upside-down, holding it at the bottom; the shape of the lock is then observed; either the lock retains its shape, which means that the lock is fixed very well; or the lock becomes deformed (under the effect of gravity) which means that the lock is not fixed very well.

The persistence with respect to water of the fixing property of the treated locks was then evaluated by immersing the treated locks in water for 5 minutes. The locks were then dried manually, followed by drying under a hood. The rigidity of the three locks was observed.

It was found that the lock treated according to Example 12 before and after immersion in water has a rigid form with good hair fixing.

The fixing of the hair of Example 12 thus shows good persistence with respect to water.

The locks treated according to Examples 10 and 11 have a rigid form before immersion in the water, but lose their rigidity after immersion in the water: the lock is supple and has no fixing.

1. A cosmetic process for treating keratin materials, comprising:
either the topical application to the keratin materials of an anhydrous (extemporaneous) mixture of a cosmetic composition comprising a maleic anhydride block polymer and of an amino alkoxysilane compound (I) or of a cosmetic composition containing same;
or the sequential application to the keratin materials of an anhydrous cosmetic composition comprising a maleic anhydride block polymer and of an amino alkoxysilane compound (I) or of an anhydrous cosmetic composition containing same, said block polymer comprising:
at least one first block with a glass transition temperature (Tg) of greater than or equal to
40° C. and obtained from at least one (meth)acrylate monomer of formula CH₂═C(R₁)—COOR₂ in which R₁ represents H or a methyl radical, R₂ represents a C₄ to C₁₂ cycloalkyl group; and
at least one second block with a glass transition temperature (Tg) of less than or equal to 20° C. and obtained from at least one maleic anhydride monomer and from at least one (meth)acrylate monomer of formula CH₂═C(R₁)—COOR₃ in which R₁ represents H or a methyl radical, R₃ representing a linear or branched C₁ to C₆ unsubstituted alkyl group, with the exception of the tert-butyl group, or a methoxyethyl group;
said amino alkoxysilane having the formula (I):
R—NH—R₁Si(OR₂)_{z}(R₃)ₓ (I)
in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R═H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3.

2. The process according to claim 1, wherein the first block of the block polymer is obtained from at least one acrylate monomer of formula CH₂═CH—COOR₂ in which R₂ represents a C₄ to C₁₂ cycloalkyl group, and from at least one methacrylate monomer of formula CH₂═C(CH₃)—COOR′₂ in which R′₂ represents a C₄ to C₁₂ cycloalkyl group;
and optionally an additional monomer chosen from linear or branched C₈-C₂₂ alkyl (meth)acrylates.

3. The process according to claim 1, wherein for the first block of the block polymer, said acrylate monomer and said methacrylate monomer are in acrylate/methacrylate mass proportions of between 30/70 and 70/30.

4. The process according to claim 2, wherein the first block of the block polymer is obtained by polymerization of isobornyl methacrylate and isobornyl acrylate.

5. The process according to claim 1, wherein the proportion of the first block in the block polymer ranges from 60% to 80%.

6. The process according to claim 1, wherein the second block of the block polymer comprises a monomer chosen from isobutyl acrylate, ethyl acrylate, n-butyl acrylate and methoxyethyl acrylate, or mixtures thereof.

7. The process according to claim 1, wherein, for the second block of the block polymer, the maleic anhydride and said (meth)acrylate monomer are in (meth)acrylate/maleic anhydride mass proportions ranging from 1 to 10.

8. The process according to claim 1, wherein the second block of the block polymer comprises an additional silicone monomer of formula (II): 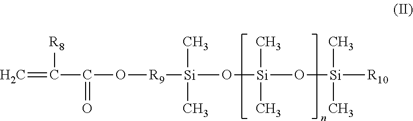
in which:
R8 denotes a hydrogen atom or a methyl group;
R9 denotes a linear or branched divalent hydrocarbon-based group containing from 1 to 10 carbon atoms and optionally containing one or two —O— ether bonds;
R10 denotes a linear or branched alkyl group containing from 1 to 10 carbon atoms;
n denotes an integer ranging from 1 to 300.

9. The process according to claim 1, wherein the proportion of the second block in the block polymer ranges from 20% to 40% by weight of the polymer.

10. The process according to claim 1, wherein said block polymer comprises an intermediate segment comprising at least one constituent monomer of the first block and at least one constituent monomer of the second block.

11. The process according to claim 1, wherein said block polymer has a polydispersity index of greater than 2.

12. The process according to claim 1, wherein at the maleic anhydride block polymer is present in the composition applied to the keratin materials in a content ranging from 0.1% to 40% by weight, relative to the total weight of the composition derived from the extemporaneous mixture.

13. The process according to claim 1, wherein, for the amino alkoxysilane (I):
R═H;
R₁ is a linear saturated C₁-C₆ hydrocarbon-based chain;
R₂ represents an alkyl group comprising from 1 to 4 carbon atoms;
R₃ represents an alkyl group comprising from 1 to 4 carbon atoms.

14. The process according to claim 1, wherein the amino alkoxysilane (I) is chosen from 3-aminopropyltriethoxysilane, 3-aminoethyltriethoxysilane, 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

15. The process according to claim 1, wherein the amino alkoxysilane (I) is used in a mole ratio of amino alkoxysilane/maleic anhydride group of the block polymer ranging from 0.01 to 10.

16. The process according to claim 1, wherein the composition comprises a hydrocarbon-based oil.

17. The process according to claim 1, wherein a mixture, prepared less than 5 minutes before application to keratin materials, of the composition comprising the maleic anhydride block polymer and of the amino alkoxysilane or of the composition containing same is applied to the keratin materials.

18. The process according to claim 1, wherein the composition comprising the maleic anhydride block polymer is first applied to keratin materials, and the amino alkoxysilane (I) or an anhydrous composition containing same and comprising a physiologically acceptable medium is then applied.

19. The process according claim 1, wherein the amino alkoxysilane (I), or an anhydrous composition containing same and comprising a physiologically acceptable medium, is first applied to keratin materials, and the composition comprising the maleic anhydride block polymer is then applied.

20. The process according to claim 1, wherein it is performed on the skin, the lips, the eyelashes, the hair or the nails.

21. A composition obtained by mixing an anhydrous composition comprising a maleic anhydride block polymer as defined in claim 1, and an amino alkoxysilane having the formula (I):
R—NH—R₁Si(OR₂)_{z}(R₃)ₓ (I)
in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R═H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3 or an anhydrous composition containing same and comprising a physiologically acceptable medium.

22. The composition according to claim 21, wherein it is a makeup composition comprising a volatile oil and a non-volatile oil, preferably a hydrocarbon-based volatile oil and a hydrocarbon-based non-volatile oil.

23. A kit comprising a first anhydrous composition comprising a maleic anhydride block polymer as defined in claim 1 and a second anhydrous composition comprising an amino alkoxysilane having the formula (I):
R—NH—R₁Si(OR₂)_{z}(R₃)ₓ (I)
in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R═H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3 and comprising a physiologically acceptable medium, the first and second compositions each being packaged in a separate packaging assembly.

24. A polymer obtained by reacting an amino alkoxysilane having the formula (I):
R—NH—R₁Si(OR₂)_{z}(R₃)ₓ (I)
in which:
R₁ is a linear or branched, saturated or unsaturated, cyclic or acyclic C₁-C₂₀ hydrocarbon-based divalent group, which may be interrupted in its chain with a heteroatom (O, S, NH) or a carbonyl group (CO), R₁ being linked to the silicon atom directly via a carbon atom;
R═H or a C₁-C₄ alkyl group, preferably H;
R₂ and R₃, which may be identical or different, represent a linear or branched alkyl group comprising from 1 to 6 carbon atoms,
z denotes an integer ranging from 1 to 3, and
x denotes an integer ranging from 0 to 2,
with z+x=3 with a maleic anhydride block polymer as defined in claim 1.
